(19) United States
(12) Patent Application Publication
(10) Pub. No.: US 2026/0301966 A1
(43) Pub. Date: Oct. 1, 2026

(54) ESTIMATION APPARATUS

(71) Applicant: NTT DOCOMO, INC., Tokyo (JP)

(72) Inventors: Syouta KOBAYASHI, Chiyoda-ku (JP); Takafumi YAMAUCHI, Chiyoda-ku (JP); Satoshi HIYAMA, Chiyoda-ku (JP)

(73) Assignee: NTT DOCOMO, INC., Tokyo (JP)

(21) Appl. No.: 19/477,170

(22) PCT Filed: Apr. 11, 2024
(86) PCT No.: PCT/JP2024/014666

§ 371 Date: Oct. 21, 2025

(30) Foreign Application Priority Data
Apr. 27, 2023 (JP) 2023-073320

(51) Int. Cl. G16H 50/30; G01W 1/00; G16H 10/60

(52) U.S. Cl. CPC G16H 50/30; G01W 1/00; G16H 10/60

(57) ABSTRACT

An acquirer configured to acquire at least one of: a lifelog that records a history of one or more types of specific activities that affect variations in immune function of a user; and a meteorological log that records a history of one or more types of meteorological elements at locations at which the user was present, wherein the one or more types of meteorological elements affect variations in the immune function of the user. The estimator configured to estimate an immune function index representing an index of the immune function during a predetermined period from an estimation target time point, based on at least one of: the history of the one or more types of specific activities recorded in the lifelog; and the history of the one or more types of meteorological elements recorded in the meteorological log. The estimator estimates the immune function index, based on at least one of: a history of a type of specific activity, from among the one or more types of specific activities, at a first time point that precedes the estimation target time point by a first appearance period corresponding to the type of specific activity; and a history of a type of meteorological element, from among the one or more types of meteorological elements, at a second time point that precedes the estimation target time point by a second appearance period corresponding to the type of meteorological element. The first appearance period is defined as a period from a time point at which the user has performed the type of specific activity to a time point at which variations in the immune function index, the variations being caused by the type of specific activity, become apparent. The second appearance period is defined as a period from a time point at which the user was present at a location with the type of meteorological element to a time point at which variations in the immune function index, the variations being caused by the type of meteorological element, become apparent.

### TECHNICAL FIELD

The present disclosure relates to estimation apparatuses.

### BACKGROUND ART

A known technology for managing health involves the use of a lifelog, which records a user's daily life (for example, Patent Document 1 and Patent Document 2). Attempts have been also made to construct a prediction model for predicting a user's immune status by machine learning (for example, Patent Document 3).

### RELATED ART DOCUMENTS

### Patent Documents

Patent Document 1: Japanese Patent Application Laid-Open Publication No. 2022-73115

Patent Document 2: Japanese Patent Application Laid-Open Publication No. 2022-130962

Patent Document 3: Japanese U.S. Pat. No. 7,220,822

### SUMMARY OF THE INVENTION

### Problem to be Solved by the Invention

However, none of Patent Documents 1 to 3 discloses estimation of an immune function index based on a user's log data, such as a lifelog.

An object of the present disclosure is to provide an estimation apparatus that can estimate a highly accurate immune function index based on a user's log data, such as a lifelog.

### Means of Solving the Problems

An estimation apparatus according to an aspect of the present disclosure includes: an acquirer configured to acquire at least one of: a lifelog that records a history of one or more types of specific activities that affect variations in immune function of a user; and a meteorological log that records a history of one or more types of meteorological elements at locations at which the user was present, in which, the one or more types of meteorological elements affect variations in the immune function of the user; and an estimator configured to estimate an immune function index representing an index of the immune function during a predetermined period from an estimation target time point, based on at least one of: the history of the one or more types of specific activities recorded in the lifelog; and the history of the one or more types of meteorological elements recorded in the meteorological log. The estimator estimates the immune function index, based on at least one of: a history of a type of specific activity, from among the one or more types of specific activities, at a first time point that precedes the estimation target time point by a first appearance period corresponding to the type of specific activity; and a history of a type of meteorological element, from among the one or more types of meteorological elements, at a second time point that precedes the estimation target time point by a second appearance period corresponding to the type of meteorological element. The first appearance period is defined as a period from a time point at which the user has performed the type of specific activity to a time point at which variations in the immune function index, the variations being caused by the type of specific activity, become apparent. The second appearance period is defined as a period from a time point at which the user has been present at a location with the type of meteorological element to a time point at which variations in the immune function index, the variations being caused by the meteorological element, become apparent.

### EFFECT OF THE INVENTION

According to one aspect of the present disclosure, the trend of immune function can be estimated with a high accuracy based on user's log data, such as a lifelog.

### BRIEF DESCRIPTION OF THE DRAWINGS

FIG. 1 is a diagram illustrating an example of a configuration of an information providing system according to an embodiment of the present disclosure.

FIG. 2 is an explanatory diagram of an immune function trend.

FIG. 3 is a diagram illustrating an example of an electrical configuration of a user apparatus.

FIG. 4 is a diagram illustrating an example of an electrical configuration of an immune function-trend information providing apparatus.

FIG. 5 is a diagram schematically illustrating an example of learning data used to learn an estimation model.

FIG. 6 is a matrix chart illustrating an example of an analysis result of sample data.

FIG. 7 is a diagram illustrating an example of data used to estimate an immune function trend from among a history of specific activities.

FIG. 8 is a conceptual diagram of grouped machine learning applied to learning data.

FIG. 9 is an explanatory diagram of clustering of the learning data.

FIG. 10 is a diagram illustrating an example of an operation of the information providing system.

### MODES FOR CARRYING OUT THE INVENTION

### 1. Embodiment

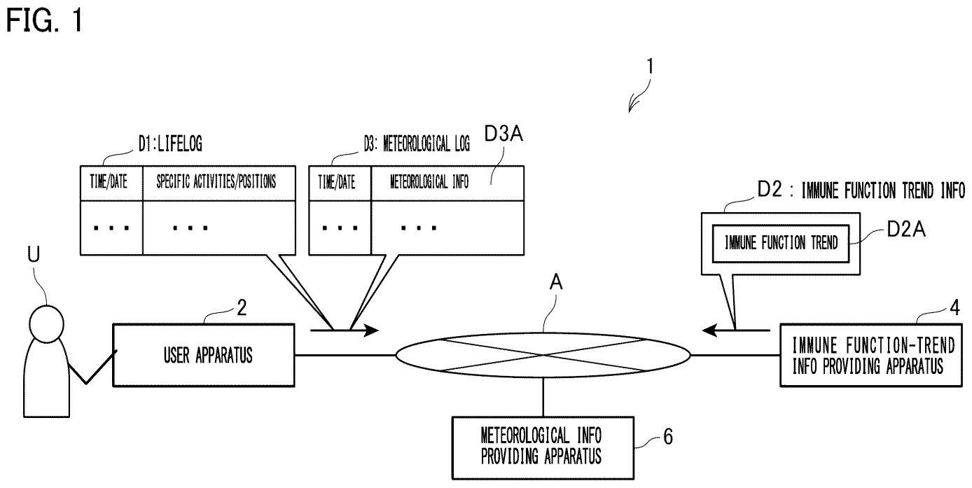

FIG. 1 is a diagram illustrating an example of a configuration of an information providing system 1 according to an embodiment of the present disclosure. The information providing system 1 estimates an immune function trend D2A of a user U based on the user's lifelog D1 and provides the user U with immune function trend information D2 including results of the estimation of the immune function trend D2A. The immune function trend D2A is an example of an “immune function index” in the present disclosure. The information providing system 1 of this embodiment also uses a meteorological log D3 that records meteorological information D3A to estimate the immune function trend D2A. In this embodiment, description will be given of the information providing system 1 that provides services through a network A.

The lifelog D1 is data that records activities in a daily life of the user U. The lifelog D1 includes information that identifies an activity history, indicating what activity the user U performed, when the activity was performed, and the duration of the activity. Specifically, each activity of the user U is associated with the date and time of performance and is recorded in the lifelog DI. The activities recorded in the lifelog DI affect the immune function trend D2A. Specifically, the lifelog D1 includes activities categorized as lifestyle habits. Examples of the activities include sleeping, walking, exercising, going out, returning home, commuting, eating, drinking alcohol, bathing, and smoking. The “activities categorized as lifestyle habits” are hereinafter referred to as “specific activities.” In this embodiment, each specific activity is associated with the date and time and is recorded in the lifelog D1.

Location information of the user U is also associated with the date and time and is recorded in the lifelog D1. These records enable identification of when and where the user U was present, as well as the duration of the stay. The following information can also be identified based on these records, a location of user U's residence, and other locations included in the location information: time user U spent at home, time the user U spent outside the home; travel time and distance between a first location and a second location; and time of returning home.

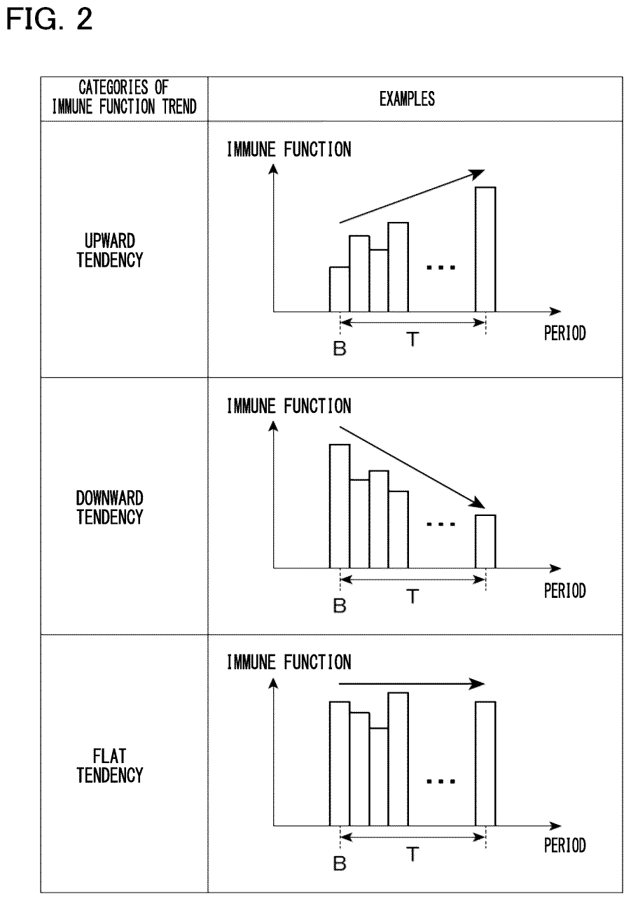

FIG. 2 is an explanatory diagram of the immune function trend D2A. The immune function trend D2A refers to a trend of variations in immune function of the user U during a predetermined period T from an estimation target time point B. In this embodiment, as illustrated in FIG. 2, the immune function trend D2A is classified into three categories: upward tendency, downward tendency, and flat tendency. The upward tendency refers to a trend of immune function increasing over the predetermined period T from the estimation target time point B. The downward tendency refers to a trend of immune function decreasing over the predetermined period T from the estimation target time point B. The flat tendency refers to a trend of immune function nearly unchanged over the predetermined period T from the estimation target time point B.

An appropriate unit, for example, an hour, a day, a week, and a month, is used as the unit of the predetermined period T. The duration of the predetermined period T may be set to an appropriate unit, such as a single unit, or multiple units. Specifically, the duration of the predetermined period T may be set to one day, two days, one week, two weeks, one month, two months, or other period lengths.

The estimation target time point B refers to a point in time at which the immune function trend D2A is to be estimated. The estimation target time point B may correspond to the current time or current day at the time of estimation, which is when the immune function trend estimation is to be performed by the information providing system 1. Alternatively, the estimation target time point B may correspond to a freely selected point in the future relative to the time of estimation execution, such as one week later, or one month later. Thus, the information providing system 1 estimates the immune function trend D2A over a predetermined period T, which is freely selectable, using the estimation target time point B as the reference time point. The estimation target time point B corresponds to either the time of estimation execution or a freely selected future time point. For example, when the estimation target time point B is set to the current day at the time of estimation, the predetermined period T is set in units of one day, then the information providing system 1 estimates the immune function trend D2A from the current day, which serves as the reference time point, to the next day.

With reference to FIG. 1, the meteorological information D3A is associated with meteorological elements at locations of the user U and affects the immune function trend D2A. The meteorological elements represent meteorological situations and phenomena. Examples of the meteorological elements that affect the immune function trend D2A include temperature, humidity, atmospheric pressure, wind speed, sunshine, cloud cover, precipitation amount, snowfall amount, snow cover amount, and weather. Humidity and temperature are known to be environmental factors that affect the growth of viruses responsible for infections. Accordingly, the humidity and the temperature are estimated to affect the immune function trend D2A.

In this embodiment, the meteorological information D3A is associated with the date and time and is recorded in the meteorological log D3. Accordingly, the meteorological log D3 enables identification of when the user U was present, the type of meteorological element, and the duration of the stay. In other words, the meteorological log D3 enables identification of a history of meteorological elements corresponding to the locations at which the user U was present. The frequency and the timing at which the meteorological information D3A is recorded in the meteorological log D3 is freely selectable.

The lifelog D1 and the meteorological log D3 may be collectively recorded in one piece of log data.

The information providing system 1 of this embodiment includes a user apparatus 2, an immune function-trend information providing apparatus 4, and a meteorological information providing apparatus 6 as illustrated in FIG. 1. Each of the user apparatus 2, the immune function-trend information providing apparatus 4, and the meteorological information providing apparatus 6 accesses the network A, such as the Internet, to establish communication via the network A. The immune function-trend information providing apparatus 4 is an example of an “estimation apparatus” in the present disclosure. Only one user apparatus 2 is illustrated in FIG. 1; however, more than one user apparatus 2 may be included in the system.

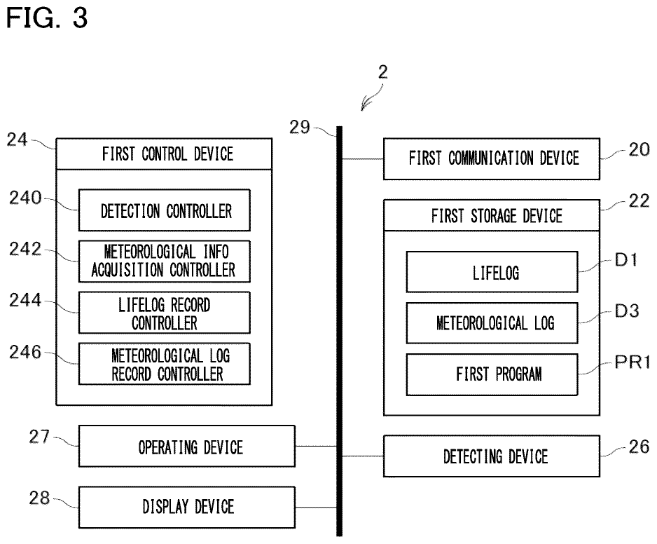

FIG. 3 is a diagram illustrating one example of an electrical configuration of the user apparatus 2. The user apparatus 2 is a portable electronic device that sequentially records the lifelog D1 and the meteorological log D3. The user apparatus 2 transmits the lifelog DI and the meteorological log D3 to the immune function-trend information providing apparatus 4 via the network A. Examples of the user apparatus 2 include a mobile phone, a smartphone, and a wearable device.

As illustrated in FIG. 3, the user apparatus 2 includes a first communication device 20, a first storage device 22, a first control device 24, a detecting device 26, an operating device 27, and a display device 28. Each device is connected to a bus 29, through which signals are transmitted and received.

The first communication device 20 is hardware that accesses the network A and includes a transmission circuit and a reception circuit.

The first storage device 22 is a recording medium readable by the first control device 24. The first storage device 22 includes a nonvolatile memory and a volatile memory. The nonvolatile memory may be a ROM (Read Only Memory), an EPROM (Erasable Programmable Read Only Memory), or an EEPROM (Electrically Erasable Programmable Read Only Memory). The volatile memory may be a RAM (Random Access Memory).

The first control device 24 includes one or more processors, such as a CPU (Central Processing Unit). For a part of the function of the first control device 24, a circuit such as a DSP (Digital Signal Processor), an ASIC (Application Specific Integrated Circuit), a PLD (Programmable Logic Device), and an FPGA (Field Programmable Gate Array) may be used instead of a CPU or in addition to a CPU.

The operating device 27 receives a user U's input. The display device 28 displays a variety of information.

The detecting device 26 detects activities of the user U. The detecting device 26 includes one or more sensors required for measurement. The detecting device 26 of this embodiment includes at least a first sensor that detects walking and exercise of the user U, a second sensor that detects sleep, and a third sensor that detects location information. The first sensor may be an acceleration sensor. The second sensor may be a pulse wave sensor and a heart rate sensor. The third sensor receives a signal including location information that indicates the location of the user apparatus 2. Examples of the signal including location information are a GNSS (an abbreviation of Global Navigation Satellite System) signal, a radio wave emitted from a base station included in a mobile communication network, and a beacon signal.

The detecting device 26 may include other sensors that measure or recognize environmental conditions. Examples of the sensors include a fourth sensor that recognizes images and videos, a fifth sensor that recognizes voice and external sounds, and a sixth sensor that recognizes use of the user apparatus 2 by the user U and a history of the use.

In this embodiment, the first storage device 22 stores therein the lifelog DI and the meteorological log D3, and a first program PR1. The first program PRI controls the user apparatus 2. The first control device 24 executes the first program PR1 to act as a detection controller 240, a meteorological information acquisition controller 242, a lifelog record controller 244, and a meteorological log record controller 246.

The detection controller 240 acts to control the detecting device 26 to sequentially acquire measurement values from the detecting device 26 and identify at least the specific activities, from among activities of the user U, based on the measurement values. The detection controller 240 also sequentially acquires location information based on the measurement values. The timing and the frequency at which the detection controller 240 acquires the measurement values are freely selectable.

The meteorological information acquisition controller 242 acts to acquire the meteorological information D3A at a location indicated by location information based on the location information included in the measurement values obtained by the detecting device 26. In this embodiment, the meteorological information acquisition controller 242 controls the first communication device 20 to acquire the meteorological information D3A from the meteorological information providing apparatus 6 through the network A. The meteorological information providing apparatus 6 is a network node that provides services of distributing the meteorological information D3A through the network A. The detecting device 26 may include a seventh sensor that detects meteorological phenomena, and the meteorological information acquisition controller 242 may acquire the meteorological information D3A from the seventh sensor. The seventh sensor may be a temperature sensor or a humidity sensor.

The lifelog record controller 244 acts to associate the specific activities and the location information identified by the detection controller 240 with the date and time.

The meteorological log record controller 246 acts to associate the meteorological information D3A acquired by the meteorological information acquisition controller 242 with the date and time and record the association in the meteorological log D3.

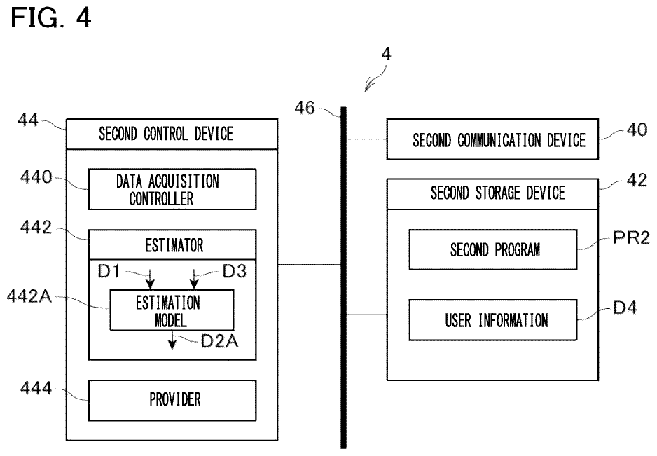

FIG. 4 is a diagram illustrating an example of an electrical configuration of the immune function-trend information providing apparatus 4. As illustrated in FIG. 4, the immune function-trend information providing apparatus 4 includes a second communication device 40, a second storage device 42, and a second control device 44. Each device is connected to a bus 46, through which signals are transmitted and received.

The second communication device 40 is hardware that accesses the network A and includes a transmission circuit and a reception circuit.

The second storage device 42 is a recording medium readable by the second control device 44. The second storage device 42 includes a non-volatile memory and a volatile memory. The non-volatile memory may be a ROM, an EPROM, or an EEPROM. The volatile memory may be a RAM.

The second control device 44 includes one or more processors, such as a CPU. For a part of the function of the second control device 44, a circuit, such as a DSP, an ASIC, a PLD, and an FPGA, may be used instead of a CPU or in addition to a CPU.

In this embodiment, the second storage device 42 stores therein a second program PR2 and user information D4.

The user information D4 is associated with the user U and includes attribute information and information used to identify lifestyle habits. The attribute information includes age, sex, address, and family composition. The information used to identify lifestyle habits includes information indicative of a residence location and other locations. For example, the information indicative of a residence location is used to identify whether the user has gone out. For example, the information indicative of other locations is used to identify movement to a location, and the duration of the stay at that location.

The second program PR2 controls the immune function-trend information providing apparatus 4. The second control device 44 executes the second program PR2 to act as a data acquisition controller 440, an estimator 442, and a provider 444. These functions are described in detail below.

The data acquisition controller 440 is an example of an “acquirer” in the present disclosure. The data acquisition controller 440 controls the second communication device 40 to acquire the lifelog D1 and the meteorological log D3 from the user apparatus 2 through the network A.

The estimator 442 estimates the immune function trend D2A based on the lifelog D1. The estimator 442 of this embodiment estimates the immune function trend D2A based on the meteorological log D3 and the type of lifestyle habits of the user U in addition to the lifelog D1. Details of the estimator 442 will be described later.

The provider 444 controls the second communication device 40 to transmit the immune function trend information D2 including an estimated result of the immune function trend D2A to the user apparatus 2 through the network A.

The estimator 442 estimates the immune function trend D2A using artificial intelligence. Specifically, the estimator 442 includes an estimation model 442A. The estimation model 442A is a trained model that is constructed in advance by machine learning and is a technique for estimating the immune function trend D2A. An appropriate method may be used in the machine learning. Examples of the use in the machine learning include a classical linear model, an SVM (Support Vector Machine), XGBoost (extreme Gradient Boosting), and a LightGBM (Light Gradient Boosting Machine). Deep learning, such as a DNN (Deep Nural Network), may be also used in the machine learning.

The estimation model 442A is a machine learning model that receives at least one of the following as an input: (i) a history of one or more types of specific activities obtained from the lifelog D1 of the user U, and (ii) a history of one or more types of meteorological elements obtained from the meteorological log D3. The estimation model 442A outputs the immune function trend D2A of the user U. The estimation model 442A may optionally receive the attribute information included in the user information D4 as an additional input. The estimation model 442A is exemplified by a machine learning model that receives the following as an input and outputs the immune function trend D2A of the user U: (i) a history of one or more types of specific activities, and (ii) a history of one or more types of meteorological elements as an input.

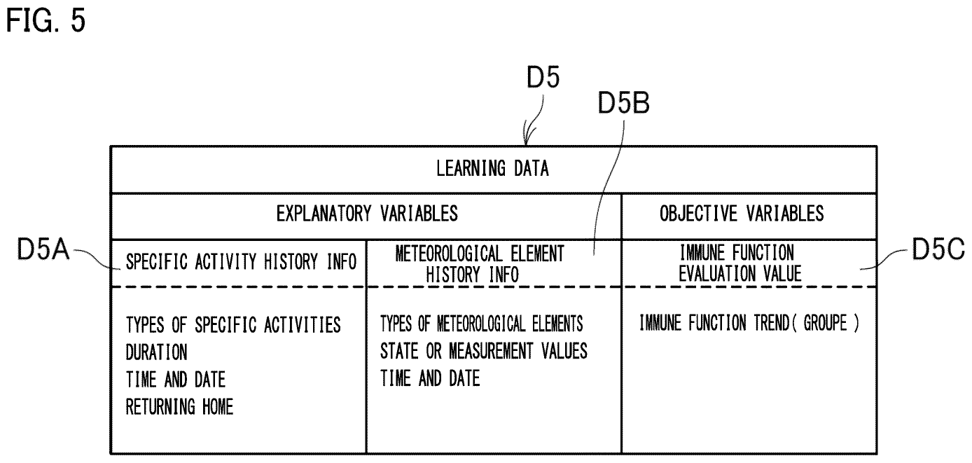

FIG. 5 is a diagram schematically illustrating an example of learning data D5 used to learn the estimation model 442A. The learning data D5 is a data set used to learn a relationship between the following: (i) at least one of a history of one or more types of specific activities and a history of one or more types of meteorological elements, which serve as explanatory variables, and (ii) the immune function trend D2A, which serves as an objective variable. In the learning data D5, the attribute information included in the user information D4 may be additionally used as an explanatory variable. The learning data D5 illustrated in FIG. 5 includes the following as explanatory variables: (i) a learning history of one or more types of specific activities, and (ii) a learning history of one or more types of meteorological elements. Specifically, the learning data D5 includes learning specific activity history information D5A and learning meteorological element history information D5B, which serve as explanatory variables. The learning data D5 illustrated in FIG. 5 also includes a learning immune function evaluation value D5C, which serves an objective variable.

The specific activity history information D5A is associated with a history of specific activities performed by the user U. The specific activity history information D5A includes information identifying the type of specific activity performed by the user U, when the specific activity was performed, and the duration of the specific activity. That is, the specific activity history information D5A includes the types of specific activities, a duration of each type of specific activity, and the date and time. In the specific activity history information D5A of this embodiment, the types of specific activities include sleep, walking, exercise, going out, and returning home.

The meteorological element history information D5B is associated with a history of meteorological elements experienced by the user U. The meteorological element history information D5B includes information identifying when the user U was present in an environment corresponding to a meteorological element, and the duration of the stay. The meteorological element history information D5B includes the types of meteorological elements, the state or measurement value of each meteorological element, the duration of the stay, and the date and time. As illustrated in FIG. 5, the meteorological elements of the meteorological element history information D5B are two types, humidity and temperature.

The immune function evaluation value D5C identifies an immune function index (the immune function trend D2A in this embodiment) that actually occurred in the user U, based on the following: (i) a history of types of specific activities identified by the specific activity history information D5A, and (ii) a history of types of meteorological elements identified by the meteorological element history information D5B. For example, the immune function evaluation value D5C may be derived from one of the following: (i) measured values of a variety of indexes used in quantitative evaluation of the immune function, (ii) a value approximated by fitting of the measured values, and (iii) an interpolated value of the measured values. The immune function evaluation value D5C may itself be the immune function trend D2A that actually occurred.

The inventors obtained two findings, a first finding and a second finding, by collecting and analyzing sample data. This data included the specific activity history information D5A, the meteorological element history information D5B, and daily immune function from a large number of human subjects.

### (First Finding)

A first appearance period El varies depending on the type of specific activity. The first appearance period El is defined as a period from a time point at which a human subject has performed the specific activity to a time point at which variations in the immune function index (the immune function trend D2A in this embodiment), caused by the specific activity, become apparent. In other words, the first appearance period El is defined as a period from the time point at which the user U has performed the type of specific activity to the time point at which variations in the immune function index, caused by the specific type of activity, become apparent.

Similarly, a second appearance period E2 varies based on the type of meteorological element. The second appearance period E2 is defined as a period from a time point at which a human subject has been present in an environment represented by the meteorological element to a time point at which variations in the immune function index, caused by the meteorological element, become apparent. In other words, the second appearance period E2 is defined as a period from the time point at which the user U has been present at a location with the type of meteorological element to the time point at which variations in the immune function index, caused by the type of meteorological element, become apparent. Hereinafter, in some cases, the first appearance period El and the second appearance period E2 are referred to as “appearance period E” without any distinction.

### (Second Finding)

Variations in the immune function index (the immune function trend D2A in this embodiment) correlate with a statistic (hereinafter, “first statistic”) related to specific activities. The degree to which variations in the immune function index obviously become appearance (hereinafter, “degree of detectability”) depends on the duration (a statistical period) used to calculate the first statistic. The duration during which the degree of detectability of variations in the immune function index is maximized varies based on the type of specific activity. Similarly, variations in the immune function index correlate with a statistic (hereinafter, “second statistic”) related to meteorological elements. The degree of detectability of variations in the immune function index depends on the duration (a statistical period) used to calculate the second statistic. The duration during which the degree of detectability of variations in the immune function index is maximized varies based on the type of meteorological element. In this example, the statistic represents either an average or a standard deviation. Hereinafter, the selected duration used to calculate the first statistic is referred to as “first selected duration F1.” The selected duration used to calculate the second statistic is referred to as “second selected duration F2.” In some cases, the first selected duration F1 and the second selected duration F2 are referred to as “selected duration F” without any distinction.

Thus, the first selected duration F1 refers to a statistical period during which the degree of detectability of variations in the immune function index, with respect to the statistic (the first statistic) of a specific type of activity, is maximized. The second selected duration F2 refers to a statistical period during which the degree of detectability of variations in the immune function index, with respect to the statistic (the second statistic) of a type of meteorological element, is maximized.

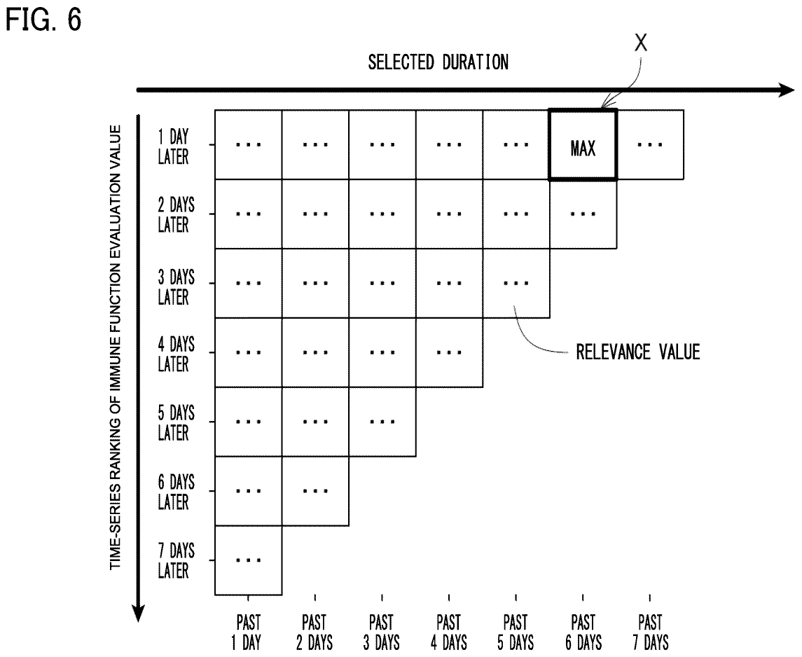

FIG. 6 is a matrix chart illustrating an example of an analysis result of sample data. The analyzed sample data includes the specific activity history information D5A and the immune function evaluation value DSC for each day. The specific activity history information D5A includes appropriate specific activities described above. The immune function evaluation value D5C includes an appropriate index value to be used for quantitative evaluation of the immune function. Examples of the immune function evaluation value D5C include the concentration of IgA, the secretion rate or secretion volume thereof, a quantification index value of immunoglobulin in the blood, and a quantification index value of immune cells.

The inventors conducted an analysis of the sample data by obtaining an evaluation value of a relationship between daily specific activities identified by the specific activity history information D5A, the selected duration F used to calculate statistics of the specific activities, and the immune function evaluation value D5C. A known or common appropriate method is used to evaluate the relationship.

The matrix chart illustrated in FIG. 6 indicates an evaluation value of the relationship for each of combinations of a time-series ranking of the immune function evaluation value DSC and the selected duration F. The vertical axis corresponds to the time-series ranking of the immune function evaluation value D5C. The horizontal axis corresponds to the selected duration F. The time-series ranking indicates a ranking in time series of the immune function evaluation value DSC for each day used to evaluate the relationship. The beginning date of the time-series ranking and the selected duration F is the earliest day in the history. In the matrix chart illustrated in FIG. 6, “one day later” on the vertical axis refers to a day following the earliest day, and “for one day” on the horizontal axis refers to the earliest day.

The illustrated example demonstrates that the evaluation value of the relationship reaches its maximum in a combination X, which consists of the following: the immune function evaluation value D5C with a time-series ranking of “one day later,” and the selected duration F of “six days.” Accordingly, based on the analysis result illustrated in FIG. 6, it is derived that the specific activities recorded in the specific activity history information D5A influence the formation of the immune function trend D2A on the following day. That is, the appearance period E is one day, and the selected duration F, during which the degree of detectability of variations in the immune function trend D2A are maximized, is “six days.”

The inventors obtained the appearance period E and the selected duration F for each type of specific activity and meteorological elements by conducting the analysis illustrated in FIG. 6. This analysis was applied to a variety of types of specific activities including sleep, walking, exercise, and others, as well as to meteorological elements, such as humidity and temperature.

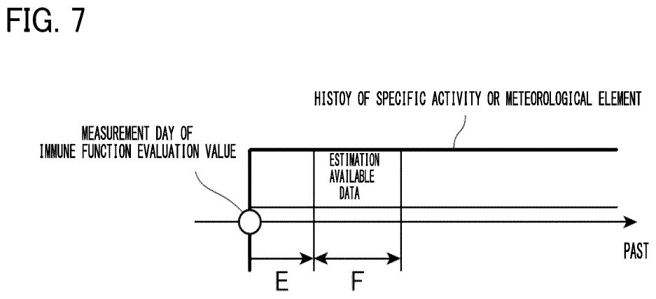

As illustrated in FIG. 7, the learning data D5 includes specific activity history information D5A, which comprises data spanning a duration corresponding to the selected duration F. This data is taken from a time point that is the appearance period E prior to the measurement day of the data serving as the basis for the immune function evaluation value DSC, with the time point determined according to the type of specific activity. Similarly, as illustrated in FIG. 7, the learning data D5 includes the meteorological element history information D5B, which comprises data spanning a duration corresponding to the selected duration F. This data is taken from a time point that is the appearance period E prior to the measurement day of the data serving as the basis for the immune function evaluation value DSC, with the time point determined according to the type of meteorological element. In other words, the specific activity history information D5A and the meteorological element history information D5B comprise data taken from a time point that is the appearance period E prior to the measurement day of the data serving as the basis for the immune function evaluation value D5C, with the data spanning the selected duration F immediately preceding that time point.

In still other words, the learning data D5 includes the learning immune function evaluation value DSC, which is an evaluation value of the immune function index. The learning data D5 includes at least one of the following: (i) the specific activity history information D5A, which serves as a learning history of a type of specific activity, and (ii) the meteorological element history information D5B, which serves as a learning history of a type of meteorological element. The specific activity history information D5A includes a history of a type of specific activity at a third time point, which proceeds the measurement day of the immune function index by an appearance period E (the first appearance period E1) corresponding to the type of specific activity. The history of a type of specific activity at the third time point includes a history of a type of specific activity in a third selected duration preceding the third time point. The third selected duration is a statistical period during which the degree of detectability of variations in the immune function index, with respect to the statistic of a type of specific activity, is maximized. The third selected duration may correspond to the first selected duration F1. Similarly, the meteorological element history information D5B includes a history of a type of meteorological element at a fourth time point, which precedes the measurement day of the immune function index by an appearance period E (second appearance period) corresponding to that type of meteorological element. The history of a type of meteorological element at the fourth time point includes a history of a type of meteorological element in a fourth selected duration preceding the fourth time point. The fourth selected duration is a statistical period during which the degree of detectability of variations in the immune function index, with respect to the statistic of a type of meteorological element, is maximized. The fourth selected duration may correspond to the second selected duration F2.

By applying machine learning to the learning data D5, an estimation model 442A is constructed. This results in a trained model that accurately captures the relationship between the history of specific activities, the history of meteorological elements, and the immune function trend D2A.

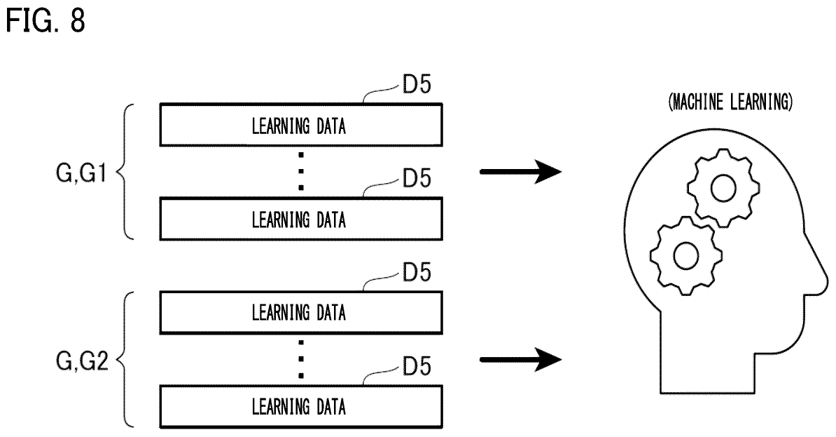

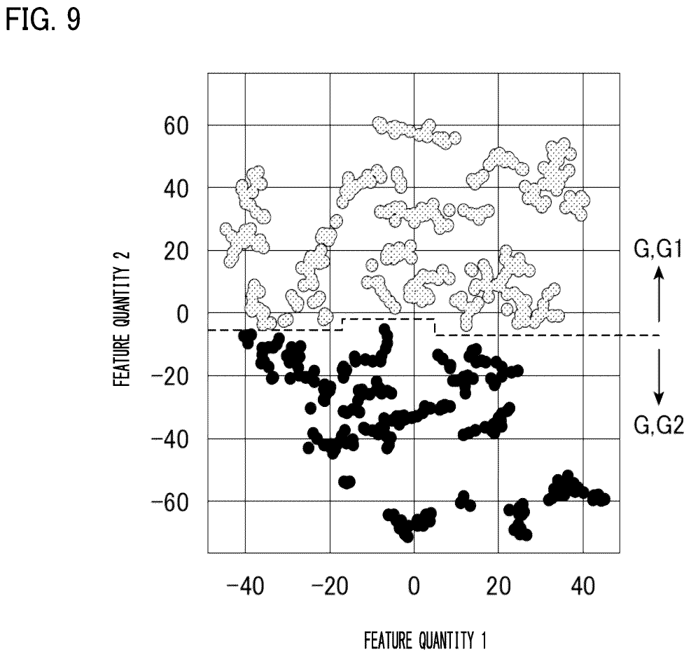

FIG. 8 is a conceptual diagram of grouped machine learning applied to the learning data D5. FIG. 9 is an explanatory diagram of clustering of the learning data D5. In this embodiment, the learning data D5 is divided into one or more groups G based on similarity of lifestyle habits of human subjects from whom the learning data D5 originates. The learning data D5 is subjected to machine learning relative to each group G. Grouping is performed, for example, by the following procedure. First, at least three feature quantities indicating the lifestyle habits of human subjects are compressed into two dimensions by using an appropriate dimension reduction algorithm, thereby converting a distribution of lifestyle habits of each human subject to a two-dimensional surface distribution as illustrated in FIG. 9. Subsequently, the human subjects are grouped into units having similar lifestyle habits by using an appropriate clustering method. Based on the results of the clustering, the learning data D5 is classified into one or more groups G. For example, t-SNE (Stochastic Neighbor Embedding) may be used as the dimension reduction algorithm. As the clustering method, a K-means method may be used.

In the example illustrated in FIG. 8, a large number of pieces of learning data D5 are classified into two groups, that is, a group G1 and a group G2, by the aforementioned grouping process. The group G1 is a cluster of learning data D5 obtained from human subjects who are relatively active. The group G2 is a cluster of learning data D5 obtained from human subjects who do not belonging to the group G1. In this case, feature quantities indicative of the lifestyle habits of human subjects may include sleep duration, number of steps walked, walking speed, walking duration, exercise duration, time spent at home, time spent outside the home, number of places visited during outings, and time of returning home.

In this case, the learning data D5 in group G1 predominantly include data obtained from human subjects having an established exercise routine, whereas the learning data D5 in group G2 predominantly include data obtained from human subjects with little or no exercise habits. Generally, it is considered that exercise in individuals with an established exercise routine leads to an increase in IgA concentration. Conversely, in individuals lacking an exercise routine, exercise is considered to induce fatigue and result in a decrease in IgA concentration.

More specifically, for example, a specific activity related to exercise, such as engaging in exercise for a prolonged period, walking a large number of steps, or visiting many locations, leads to an increase in IgA concentration in individuals with an established exercise routine. Accordingly, such activity becomes a factor contributing to the formation of the immune function trend D2A exhibiting an “upward tendency.” Conversely, in individuals lacking such habits, these same activities may result in reduced IgA concentrations, thereby contributing to the formation of an immune function trend D2A characterized by a downward tendency.

The estimation model 442A is constructed by using machine learning applied to the learning data D5 for each of the group GI and the group G2. Accordingly, for example, in response to an input representing a specific activity related to exercise performed by a user U, the immune function trend D2A can be accurately output based on whether the user U is a relatively active individual.

As shown in FIG. 4, the estimator 442 includes estimation models 442A respectively corresponding to each of one or more groups G. The estimator 442 estimates the immune function trend D2A using the estimation model 442A that corresponds to the type of lifestyle habits of the user U. The type of lifestyle habits indicates a group G in which the lifestyle habits of the user U are similar, that is, a classification based on the similarity of users' lifestyle habits. The estimator 442 identifies the type of lifestyle habits based on the user information D4. Alternatively, the estimator 442 may use the lifelog D1 in place of the user information D4 to identify the type of lifestyle habits.

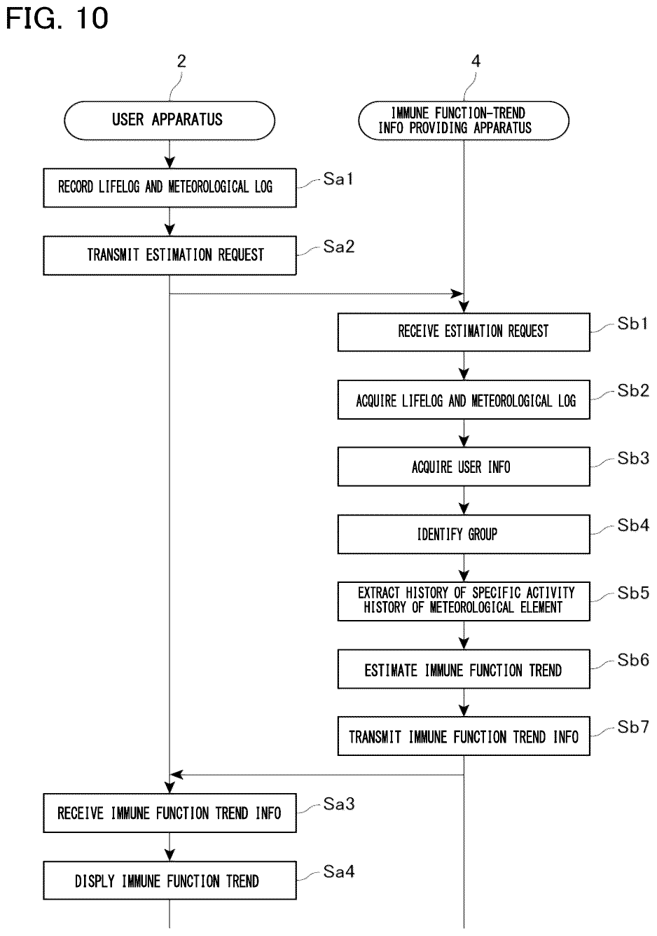

FIG. 10 is a diagram illustrating an example of the operation of the information providing system 1. In this example of the operation, the immune function-trend information providing apparatus 4 estimates the immune function trend D2A based on the lifelog D1, the meteorological log D3, and the type of lifestyle habits of the user U.

First, the user apparatus 2 sequentially records the lifelog D1 and the meteorological log D3, and stores them in the first storage device 22 (Step Sa1). Next, when the user U desires to obtain the immune function trend D2A, a predetermined input operation is performed on the user apparatus 2. Upon receiving the predetermined operation, the user apparatus 2 transmits an estimation request for the immune function trend D2A to the immune function-trend information providing apparatus 4 (Step Sa2).

When the immune function-trend information providing apparatus 4 receives the estimation request from the user apparatus 2 (Step Sb1), the data acquisition controller 440 acquires the lifelog DI and the meteorological log D3 of the user U (Step Sb2). The estimator 442 acquires the user information D4 of the user U from the second storage device 42 (Step Sb3). The estimator 442 acquires the user information and determines whether the user U belongs the group G1 or G2 based on the lifestyle habits of the user U indicated by the user information D4, thereby identifying the type of lifestyle habits of the user U (Step Sb4). As described above, the estimator 442 may use the lifelog D1 in place of the user information D4 to identify the type of lifestyle habits.

The estimator 442 extracts a history of a type of specific activity from the lifelog D1 and extracts also a history of a type of meteorological element obtained from the meteorological log D3 (Step Sb5). At this Step Sb5, the estimator 442 extracts a history of a type of specific activity at a first point time preceding “this day” as an estimation target time point B by the appearance period E (the first appearance period El) corresponding to the type of specific activity. The estimator 442 extracts a history of a type of specific activity in the selected duration F (the first selected duration F1) preceding the first time point from the history of the type of specific activity at the first time point. At Step Sb5, the estimator 442 extracts a history of a type of meteorological element at a second time point that precedes “this day” as the estimation target time point B by the appearance period E (the second appearance period E2) corresponding to the type of meteorological element. The estimator 442 extracts a history of the type of specific activity in the selected duration F (the second selected duration F2) preceding the second time point from the history of the type of meteorological element at the second time point.

The estimator 442 then inputs a history of various types of specific activities and a history of various types of meteorological elements to the estimation model 442A corresponding to the type of lifestyle habits, thereby obtaining an estimated result of the immune function trend D2A from the estimation model 442A (step Sb6).

Next, the provider 444 transmits the immune function trend information D2 including the estimated result of the immune function trend D2A to the user apparatus 2 (Step Sb7). The estimator 442 may identify lifestyle habits as a basis of the estimation of the immune function trend D2A. For example, when the “downward tendency” is estimated as the immune function trend D2A, the estimator 442 identifies lifestyle habits having contributed to the “downward tendency.” For example, an interpretation tool method, such as LIME (Local Interpretable Model-agnostic Explanations) and SHAP (SHapley Additive explanations), may be used to identify lifestyle habits.

Upon receipt of the immune function trend information D2 (Step Sb8), the user apparatus 2 displays the immune function trend D2A to present the immune function trend D2A to the user U (Step Sb9). This display enables the user U to understand the immune function trend D2A.

As described above, the immune function-trend information providing apparatus 4 of this embodiment includes the data acquisition controller 440 configured to acquire the lifelog DI that records a history of one or more types specific activities that affect variations in the immune function of the user U, and the estimator 442 configured to estimate the immune function trend D2A indicative of a tendency of variations in the immune function during the predetermined period T from the estimation target time point B based on a history of a type of specific activity. The estimator 442 estimates the immune function trend D2A based on a history of a type of specific activity at a first time point that precedes the estimation target time point B by the first appearance period El. The appearance period E varies depending on the type of specific activity.

With this configuration, the appearance period E during which a type of specific activity contributes to formation of the immune function trend D2A is reflected in estimation of the immune function trend D2A. Accordingly, the immune function trend D2A can be accurately estimated based on the history of a type of specific activity.

In the immune function-trend information providing apparatus 4 of this embodiment, the estimator 442 estimates the immune function trend D2A based on the history of a type of specific activity for the selected duration F (the first selected duration F1) that precedes the first time point. The first selected duration F1 corresponds to a statistical period during which the degree of detectability of variations in the immune function trend D2A, with respect to the statistic of a type of specific activity, is maximized.

With this configuration, the history in the period during which the degree of detectability of variations in the immune function trend D2A is maximized is used to estimate the immune function trend D2A. Accordingly, the immune function trend D2A is more accurately estimated.

In this embodiment, the data acquisition controller 440, included in the immune function-trend information providing apparatus 4, acquires a meteorological log D3, which is a history of meteorological elements at locations at which the user U was present. The meteorological log D3 includes one or more types of meteorological elements that affect variations in the immune function of the user U. The estimator 442 estimates the immune function trend D2A based on a history of a type of specific activity recorded in the lifelog DI and a history of a type of meteorological element recorded in the meteorological log D3. Furthermore, the estimator 442 estimates the immune function trend D2A based on the following:
(i) the history of a type of specific activity at the first time point that precedes the estimation target time point B by the appearance period E (the first appearance period E1) corresponding to the type of specific activity; and
(ii) a history of a type of meteorological element at a second time point that precedes the estimation target time point B by the appearance period E (the second appearance period E2) corresponding to the type of meteorological element.

With this configuration, the estimation of the immune function trend D2A utilizes not only the history of a type of specific activity but also the history of a type of meteorological element, with the appearance period E corresponding to the meteorological element also being reflected. Accordingly, the immune function trend D2A can be estimated with greater accuracy.

The estimator 442 estimates the immune function trend D2A based on the following: (i) a history of a type of specific activity spanning the selected duration F (the first selected duration F1) that precedes the first time point; and (ii) a history of a type of meteorological element spanning the selected duration F (the second selected duration F2) that precedes the second time point. The second selected duration F2 corresponds to a statistical period during which the degree of detectability of variations in the immune function trend D2A, with respect to the statistic of a type of meteorological element, is maximized.

With this configuration, the estimation of the immune function trend D2A utilizes not only the history of a type of specific activity, but also the history of a type of meteorological element, with the appearance period E corresponding to the meteorological element also being reflected. Furthermore, since the estimation uses history data spanning the period during which the degree of detectability is maximized for each type of specific activity and each type of meteorological element, the immune function trend D2A can be estimated with even greater accuracy.

In this embodiment, the estimator 442, included in the immune function-trend information providing apparatus 4, may use the attribute information of the user U to estimate the immune function trend D2A in addition to any one of the history of specific activities and the history of meteorological elements.

With this configuration, the immune function trend D2A is estimated based on the attribute of the user U.

In this embodiment, the estimator 442, included in the immune function-trend information providing apparatus 4, estimates the immune function trend D2A based on the history of specific activities, the history of meteorological elements, and lifestyle habits of the user.

With this configuration, the immune function trend D2A can be accurately estimated according to the type of lifestyle habits of the user.

In this embodiment, the estimator 442, included in the immune function-trend information providing apparatus 4, includes the estimation model 442A that learns, by using machine learning applied to the learning data D5, the relationship between (i) the history of one or more types of specific activities and the history of one or more types of meteorological elements, and (ii) the immune function trend. The learning data D5 includes the immune function evaluation value DSC, the specific activity history information D5A representing a learning history of a type of specific activity, and the meteorological element history information D5B representing a learning history of a type of meteorological element. The specific activity history information D5A includes a history of a type of specific activity at a third time point that precedes a measurement day of the immune function index by the appearance period E (the first appearance period) corresponding to the type of specific activity. The history of a type of specific activity at the third time point includes a history of the type of specific activity during a third selected duration that precedes the third time point. The third selected duration corresponds to a statistical period during which the degree of detectability of variations in the immune function index, with respect to the statistic of the type of specific activity, is maximized. The meteorological element history information D5B includes a history of a type of meteorological element at a fourth time point that precedes the measurement day of the immune function index by the appearance period E (the second appearance period) corresponding to the type of meteorological element. The history of a type of specific activity at the fourth time point includes a history of the type of meteorological element during a fourth selected duration that precedes the fourth time point. The fourth selected duration corresponds to a statistical period during which the degree of detectability of variations in the immune function index, with respect to the statistic of the type of meteorological element, is maximized.

With this configuration, machine learning is performed using histories of specific activities and meteorological elements that correspond to the respective appearance period E for each type, and further correspond to a period during which the degree of detectability is maximized. Accordingly, the estimation model 442A that accurately estimates the immune function trend D2A is obtained.

The attribute information included in the user information D4 may be additionally used as the learning data D5.

In this embodiment, the estimation model 442A, included in the immune function-trend information providing apparatus 4, is a machine-learned model that, for each cluster of the learning data D5 obtained from human subjects with similar lifestyle habits, the relationship between (i) the history of one or more types of specific activities and the history of one or more types of meteorological elements, and (ii) the immune function trend D2A.

With this configuration, the estimation model 442A is obtained that can accurately estimate the immune function trend D2A according to the types of lifestyle habits of users.

2. Modifications Modifications that may be made to the embodiment will be described below. Two or more modifications freely selected from the following may be combined as appropriate as long as they do not conflict.
(1) In the foregoing embodiment, the immune function trend D2A, representing the tendency of immune function variations, is described as an example of the “immune function index” of the present disclosure. However, the immune function index may alternatively be any appropriate indicator, such as: a degree of steepness in the increase or decrease of immune function; whether the immune function is higher or lower than the average immune function of user U; or the absolute value of the immune function. In this case, the “degree of steepness in the increase or decrease of immune function” may be evaluated based on the magnitude of the slope of the arrow illustrated in FIG. 2. Whether the immune function is higher or lower than the average immune function of user U″ may be evaluated by comparing the level of immune function indicated by the bars in the graph illustrated in FIG. 2 with the average value over a selected past period of the bar graph, or by calculating the difference between the two. The “absolute value of the immune function” may be evaluated based on the height of the bars in the graph illustrated in FIG. 2.
(2) In the foregoing embodiment, a configuration is described in which the estimator 442 of the immune function-trend information providing apparatus 4 estimates the immune function trend D2A (an example of the immune function index) based on both the lifelog DI, which records the history of one or more types of specific activities, and the meteorological log D3, which records the history of one or more types of meteorological elements. However, the estimator 442 may alternatively estimate the immune function index based on at least one of the lifelog D1 and the meteorological log D3.
(3) In the foregoing embodiment, the estimation model 442A is described as a machine learning model that learns the relationship between (i) both the history of one or more types of specific activities and the history of one or more types of meteorological elements, and (ii) the immune function index. However, the estimation model 442A may alternatively be a machine learning model that learns the relationship between (i) the immune function index and (ii) at least one of the history of one or more types of specific activities and the history of one or more types of meteorological elements.
(4) In the foregoing embodiment, as an example, the learning data D5 is classified into two groups, G1 and G2, by clustering based on lifestyle habits, and machine learning of the estimation model 442A is performed separately for each group. However, it is sufficient for the learning data D5 to be classified into at least one groups G.

That is, the number of groups G in the learning data D5 may be one, without performing clustering. Even when the number of groups G is one, the estimation model 442A can still be obtained to correspond to various lifestyle habits indicated in at least one of the history of specific activities and the history of meteorological elements. As a result, the immune function index of the user U, regardless of their lifestyle habits, can be estimated. In this case, it is not necessary to identify the user's lifestyle habit type, and therefore, the estimator 442 does not need to execute Step Sb4 in FIG. 10.

By classifying the learning data D5 into two or more groups G based on clustering according to similarities in lifestyle habits, the immune function index can be estimated with high accuracy for each group. In this case, instead of configuring the estimator 442 to include separate estimation models 442A for each of the two or more groups G, it may be configured to include a single estimation model 442A that has learned the relationship between the immune function trend D2A and at least one of the history of one or more types of specific activities and the history of one or more types of meteorological elements for each group G.
(5) In the foregoing embodiment, the user apparatus 2 may include the estimator 442 of the immune function-trend information providing apparatus 4, and the user apparatus 2 may estimate the immune function index using the estimator 442.

### 3. Other Matters

(1) In the foregoing embodiment, a read only memory (ROM) and a random access memory (RAM) are exemplified as the first storage device 22 and the second storage device 42, but the first storage device 22 and the second storage device 205 may be flexible disks, magneto-optical disks (for example, compact discs, digital versatile discs, or Blu-ray (registered trademark) discs), smart cards, flash memory devices (for example, cards, sticks, or key drives), compact disc-ROMs (CD-ROMs), registers, removable disks, hard disks, floppy (registered trademark) disks, magnetic strips, databases, servers, or other appropriate storage media.
(2) In the foregoing embodiment, the described information, signals, and the like may be represented using any of a variety of different technologies. For example, data, instructions, commands, information, signals, bits, symbols, chips, and the like that may be mentioned throughout the above description may be represented by voltages, currents, electromagnetic waves, magnetic fields or particles, optical fields or photons, or any combination thereof.
(3) In the foregoing embodiment, the input/output information and the like may be stored in a specific location (for example, a memory) or may be managed using a management table. The input/output information and the like can be overwritten, updated, or additionally written. The output information and the like may be deleted. The input information and the like may be transmitted to another device.
(4) In the foregoing embodiment, the determination may be performed using a value (0 or 1) represented by using one bit, may be performed using a Boolean value (true or false), or may be performed by comparison of numerical values (for example, comparison with a predetermined value).
(5) The order of the processing procedure, sequence, flowchart, and the like illustrated in the embodiments may be changed as long as there is no contradiction. For example, for the methods described in the present disclosure, elements of various steps are presented using an example order, and are not limited to the particular order presented.
(6) Each function illustrated in the embodiment is implemented by an arbitrary combination of at least one of hardware or software. A method for implementing each functional block is not particularly limited. That is, each functional block may be implemented by using one physically or logically combined device, or may be implemented by directly or indirectly (for example, in a wired or wireless manner) connecting two or more physically or logically separated devices and using the devices. The functional block may be implemented by combining software with the one or more devices.
(7) The program exemplified in the embodiment should be interpreted broadly to mean an instruction, an instruction set, a code, a code segment, a program code, a program, a subprogram, a software module, an application, a software application, a software package, a routine, a subroutine, an object, an executable file, an execution thread, a procedure, a function, or the like, regardless of whether the program is referred to as software, firmware, middleware, microcode, or a hardware description language, or referred to by another name.

Software, instructions, information, and the like may be transmitted and received via a transmission medium. For example, when software is transmitted from a website, server, or other remote source using at least one of a wired technology (a coaxial cable, an optical fiber cable, a twisted pair, a digital subscriber line (DSL), or the like) or a wireless technology (infrared rays, microwaves, or the like), at least one of the wired technology or the wireless technology is included within the definition of the transmission medium.
(8) In the foregoing embodiment, the terms “system” and “network” are used interchangeably.
(9) The information, the parameter, or the like described in the present disclosure may be represented by using an absolute value, may be represented by using a relative value from a predetermined value, or may be represented by using another corresponding information.
(10) In the foregoing embodiment, the terms “connected”, “coupled”, or any variation thereof, mean any direct or indirect connection or coupling between two or more elements, including the presence of one or more intermediate elements between two elements that are “connected” or “coupled” to each other. The coupling or connection between the elements may be physical coupling or connection, logical coupling or connection, or a combination thereof. For example, “connection” may also be read as “access.” When used in the present disclosure, two elements may be “connected” or “coupled” to each other using at least one of one or more wires, cables, or printed electrical connections, and using electromagnetic energy having a wavelength in a radio frequency region, a microwave region, and a light (both visible and invisible) region, and the like as some non-limiting and non-exhaustive examples.
(11) In the foregoing embodiment, the phrase “based on” does not mean “based only on” unless explicitly stated otherwise. In other words, the phrase “based on” means both “based only on” and “based at least on.”
(12) The term “determining” used in the present disclosure may encompass a wide variety of operations. The term “determining” can include, for example, judging, calculating, computing, processing, deriving, investigating, looking up, searching, inquiring (for example, looking up in a table, a database, or another data structure), and ascertaining that can be considered as “determining.” The “determining” can include receiving (for example, receiving information), transmitting (for example, transmitting information), inputting, outputting, and accessing (for example, accessing data in a memory) that can be considered as “determining.” The “determining” can include resolving, selecting, choosing, establishing, and comparing that can be considered as “determining.” That is, “determining” can include any operation that can be considered as “determining.” The “determining” may be read as “assuming”, “expecting”, “considering”, or the like.
(13) In the foregoing embodiment, when the terms “include”, “including”, and variations thereof are used, the terms are intended to be inclusive in the same manner as the term “comprising.” The term “or” used in the present disclosure is intended not to be an exclusive OR.
(14) In the present disclosure, for example, when articles such as “a”, “an”, and “the” in English are added by translation, the present disclosure may include a case in which a noun following these articles is a plural form.
(15) In the present disclosure, the expression “A and B are different” may mean that “A and B are different from each other.” The term may mean that “A and B are different from C.” The terms such as “separated”, “coupled” and the like may also be interpreted in the same manner as “different.”
(16) The foregoing aspects and embodiment in the present disclosure may be used alone, or may be used in combination, or may be switched with execution. Notification of predetermined information (for example, notification of “being X”) is not limited to notification performed explicitly, and may be performed implicitly (for example, notification of the predetermined information is not made).

### Description of Reference Signs

1 . . . information providing system, 2 . . . user apparatus, 4 . . . immunity-trend information providing apparatus, 440 . . . data acquisition controller, 442 . . . estimator, 442A . . . estimation model, 444 . . . provider, A . . . network, B . . . estimation target time point, D1 . . . lifelog, D2 . . . immune function trend information, D2A . . . immune function trend, D3 . . . meteorological log, D3A . . . meteorological information, D4 . . . user information, D5 . . . learning data, D5A . . . specific activity history information, D5B . . . meteorological element history information, D5C . . . immune function evaluation value, E . . . appearance period, F . . . selected period duration, T . . . predetermined period, U . . . user.

1. An estimation apparatus comprising:
an acquirer configured to acquire at least one of:
a lifelog that records a history of one or more types of specific activities that affect variations in immune function of a user; and
a meteorological log that records a history of one or more types of meteorological elements at locations at which the user was present, wherein the one or more types of meteorological elements affect variations in the immune function of the user; and
an estimator configured to estimate an immune function index representing an index of the immune function during a predetermined period from an estimation target time point, based on at least one of:
the history of the one or more types of specific activities recorded in the lifelog; and
the history of the one or more types of meteorological elements recorded in the meteorological log, wherein:
the estimator estimates the immune function index, based on at least one of:
a history of a type of specific activity, from among the one or more types of specific activities, at a first time point that precedes the estimation target time point by a first appearance period corresponding to the type of specific activity; and
a history of a type of meteorological element, from among the one or more types of meteorological elements, at a second time point that precedes the estimation target time point by a second appearance period corresponding to the type of meteorological element,
the first appearance period is defined as a period from a time point at which the user has performed the type of specific activity to a time point at which variations in the immune function index, the variations being caused by the type of specific activity, become apparent, and
the second appearance period is defined as a period from a time point at which the user has been present at a location with the type of meteorological element to a time point at which variations in the immune function index, the variations being caused by the type of meteorological element, become apparent.

2. The estimation apparatus according to claim 1, wherein:
the history of the type of specific activity includes a history of the type of specific activity during a first selected duration that precedes the first time point,
the first selected duration corresponds to a statistical period during which a degree of detectability of variations in the immune function index, with respect to a statistic of the type of specific activity, is maximized,
the history of the type of meteorological element includes a history of the type of meteorological element during a second selected duration that precedes the second time point, and
the second selected duration corresponds to a statistical period during which the degree of detectability of variations in the immune function index, with respect to a statistic of the type of meteorological element, is maximized.

3. The estimation apparatus according to claim 2,
wherein the estimator further uses attribute information of the user to estimate the immune function index.

4. The estimation apparatus according to claim 1,
wherein the estimator estimates the immune function index according to a type of lifestyle habits of the user.

5. The estimation apparatus according to claim 1, wherein:
the estimator includes an estimation model that learns, using machine learning applied to learning data, a relationship between an immune function index and at least one of:
a history of one or more types of specific activities; and
a history of one or more types of meteorological elements, the learning data includes:
a learning immune function evaluation value representing an evaluation value of the immune function index; and
at least one of a learning history of the type of specific activity, and a learning history of the type of meteorological element,
the learning history of the type of specific activity includes a history of the type of specific activity at a third time point that precedes a measurement day of the immune function index by the first appearance period,
the history of the type of specific activity includes a history of the type of specific activity during a third selected duration that precedes the third time point,
the third selected duration corresponds to a statistical period during which a degree of detectability of variations in the immune function index, with respect to a statistic of the type of specific activity, is maximized,
the learning history of the type of meteorological element includes a history of the type of meteorological element at a fourth time point that precedes the measurement day of the immune function index by the second appearance period,
the history of the type of meteorological element includes a history of the type of meteorological element in a fourth selected duration that precedes the fourth time point, and
the fourth selected duration corresponds to a statistical period during which the degree of detectability of variations in the immune function index, with respect to the statistic of the type of meteorological element, is maximized.

6. The estimation apparatus according to claim 5,
wherein the estimation model is a machine learning model that learns a relationship, based on lifestyle habits of human subjects, between:
the immune function index; and
at least one of the history of one or more types of specific activities and the history of one or more types of meteorological elements.
